(19) United States
(12) Patent Application Publication
(10) Pub. No.: US 2024/0099640 A1
(43) Pub. Date: Mar. 28, 2024

(54) ELECTROCARDIOGRAM ANALYSIS

(71) Applicant: Welch Allyn, Inc., Skaneateles Falls, NY (US)

(72) Inventors: Patrick James Noffke, Hartland, WI (US); Johannes de Bie, San Pietro (IT); Ryan Donlon, Milton, MA (US); Jennifer L. Ireland, Franklin, WI (US); Jeffrey D. Ryan, Syracuse, NY (US); Sharone Zlochiver, Mequon, WI (US)

(21) Appl. No.: 18/468,446

(22) Filed: Sep. 15, 2023

Related U.S. Application Data
(60) Provisional application No. 63/376,637, filed on Sep. 22, 2022.

(51) Int. Cl. A61B 5/358; A61B 5/00; A61B 5/339

(52) U.S. Cl. CPC A61B 5/358; A61B 5/339; A61B 5/7221; A61B 5/746

(57) ABSTRACT

A system for interpreting electrocardiograms receives a first electrocardiogram waveform, and identifies a feature in the first electrocardiogram waveform. The system retrieves one or more second electrocardiogram waveforms recorded at a point in time prior to the first electrocardiogram waveform. The system extracts the feature from the one or more second electrocardiogram waveforms, and generates one or more interpretive statements based on a comparison of the feature identified in the first electrocardiogram and the feature extracted from the one or more second electrocardiogram waveforms.

### BACKGROUND

An electrocardiogram (ECG) is a common diagnostic tool used to assess cardiac function. The ECG measures electrical activity of the heart from electrodes positioned at different points on a patient's body. Key features of the ECG include the P-wave, QRS complex, and T-wave, each representing a different stage of the heartbeat. These features are often analyzed for detection of abnormalities affecting the rhythm and electrical activity of the heart.

### SUMMARY

In general terms, the present disclosure relates to electrocardiogram analysis. In one possible configuration, interpretive statements are generated for a first electrocardiogram based on one or more features identified in the first electrocardiogram and the one or more features extracted from one or more second electrocardiogram waveforms recorded at a point in time prior to the first electrocardiogram waveform. Various aspects are described in this disclosure, which include, but are not limited to, the following aspects.

One aspect relates to a system for interpreting electrocardiograms, the system comprising: at least one processing device; and a memory device storing instructions which, when executed by the at least one processing device, cause the at least one processing device to: receive a first electrocardiogram waveform; identify a feature in the first electrocardiogram waveform; retrieve one or more second electrocardiogram waveforms, the one or more second electrocardiogram waveforms being recorded at a point in time prior to the first electrocardiogram waveform; extract the feature from the one or more second electrocardiogram waveforms; and generate one or more interpretive statements based on a comparison of the feature identified in the first electrocardiogram waveform and the feature extracted from the one or more second electrocardiogram waveforms.

Another aspect relates to a method of interpreting electrocardiograms, the method comprising: receiving a first electrocardiogram waveform; identifying a feature in the first electrocardiogram waveform; retrieving one or more second electrocardiogram waveforms, the one or more second electrocardiogram waveforms being recorded at a point in time prior to the first electrocardiogram waveform; extracting the feature from the one or more second electrocardiogram waveforms; and generating one or more interpretive statements based on a comparison of the feature identified in the first electrocardiogram waveform and the feature extracted from the one or more second electrocardiogram waveforms.

Another aspect relates to a computer-readable data storage medium comprising software instructions that, when executed, cause at least one computing device to: receive a first electrocardiogram waveform; identify a feature in the first electrocardiogram waveform; retrieve one or more second electrocardiogram waveforms, the one or more second electrocardiogram waveforms being recorded at a point in time prior to the first electrocardiogram waveform; extract the feature from the one or more second electrocardiogram waveforms; and generate one or more interpretive statements based on a comparison of the feature identified in the first electrocardiogram waveform and the feature extracted from the one or more second electrocardiogram waveforms.

### DESCRIPTION OF THE FIGURES

The following drawing figures, which form a part of this application, are illustrative of the described technology and are not meant to limit the scope of the disclosure in any manner.

FIG. 1 schematically illustrates an example of a system for interpreting an electrocardiogram.

FIG. 2 illustrates an example of a graphical user interface generated by the system of FIG. 1 for viewing and editing clinical reports.

FIG. 3 illustrates in more detail an example of an interpretation box displayed in the graphical user interface of FIG. 2.

FIG. 4 illustrates in more detail an example of the interpretation box of FIG. 3 after a clinician has edited interpretive statements to create an updated interpretation of the physiological data that includes physician edited interpretive statements.

FIG. 5 schematically illustrates an example of a method of interpreting electrocardiograms that can be performed by the system of FIG. 1.

FIG. 6 schematically illustrates an example of a process flow diagram for an algorithm that is executed in an operation of the method of FIG. 5.

FIG. 7 schematically illustrates another example of a method of interpreting electrocardiograms that can be performed by the system of FIG. 1.

FIG. 8 schematically illustrates an example of a system that can be used to implement the algorithm of FIG. 6.

FIG. 9 schematically illustrates another example of a system that can be used to implement the algorithm of FIG. 6.

FIG. 10 schematically illustrates another example of a system that can be used to implement the algorithm of FIG. 6.

### DETAILED DESCRIPTION

ECG acquisition devices often provide, together with graphic ECG waveforms, automatic measurements (e.g., heart rate) and a suggested textual description of the ECG findings, known as an interpretive algorithm. Currently, interpretive algorithms do not replace physician reading. Therefore, an over-reading process is often needed, introducing a delay of confirmed interpretation of 24-48 hours for the routine workflow, or requires ad-hoc consultation of a specialist in emergency care. In some instances, interpretive algorithms are erroneous causing inappropriate treatment (e.g., inappropriate prescription of anticoagulants with incorrect atrial fibrillation interpretation), inappropriate tests (e.g., inappropriate catheterization lab testing with incorrect acute myocardial infarction (AMI) interpretation), and interruptions in care.

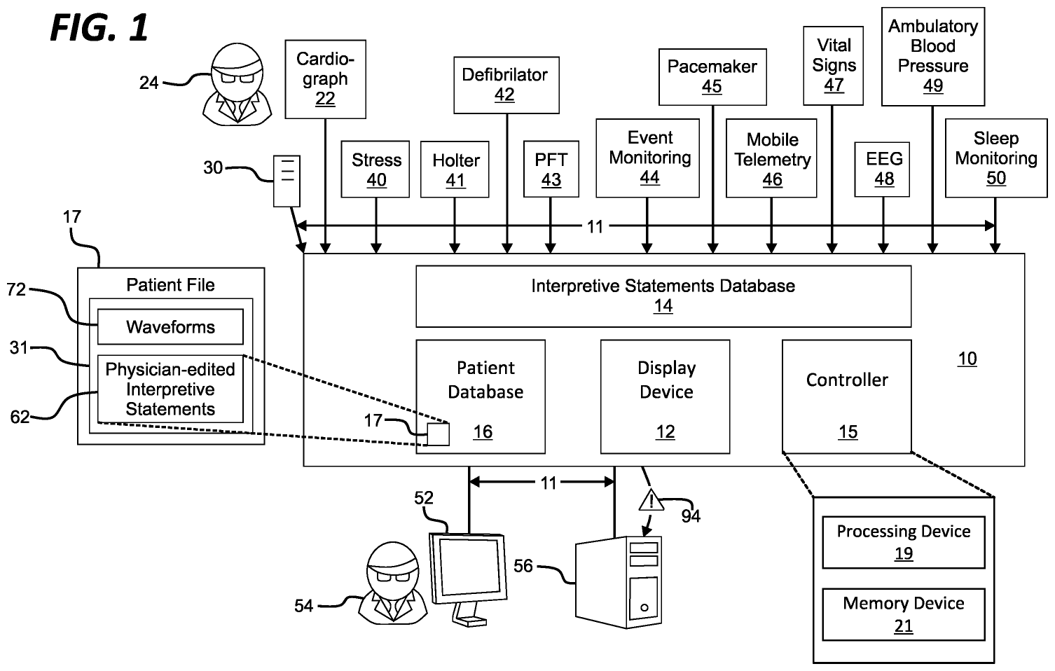

FIG. 1 schematically illustrates an example of a system 10 for analyzing and interpreting an electrocardiogram (ECG) of a patient 24. The system 10 allows a clinician 54 to view and edit one or more clinical reports 30 that include ECG recordings from the patient 24, and to trigger one or more critical alerts 94. In some instances, the system 10 includes aspects of the system described in U.S. Pat. No. 9,408,550 B2, entitled EVOLVING SERIAL COMPARISON SYSTEM WITH CRITICAL ALERT NOTIFICATIONS, issued on Aug. 9, 2016, the disclosure of which is hereby incorporated by reference in its entirety.

As shown in FIG. 1, a cardiograph device 22 generates the clinical reports 30 that include the ECG waveform of the patient 24. The cardiograph device 22 can include one or more leads that attach to a chest of the patient 24 for measuring the ECG waveform. The system 10 receives the clinical report 30 from the cardiograph device 22 via a connection such as through Wi-Fi, a local area network (LAN), the Internet, or other network, via a network interface 11.

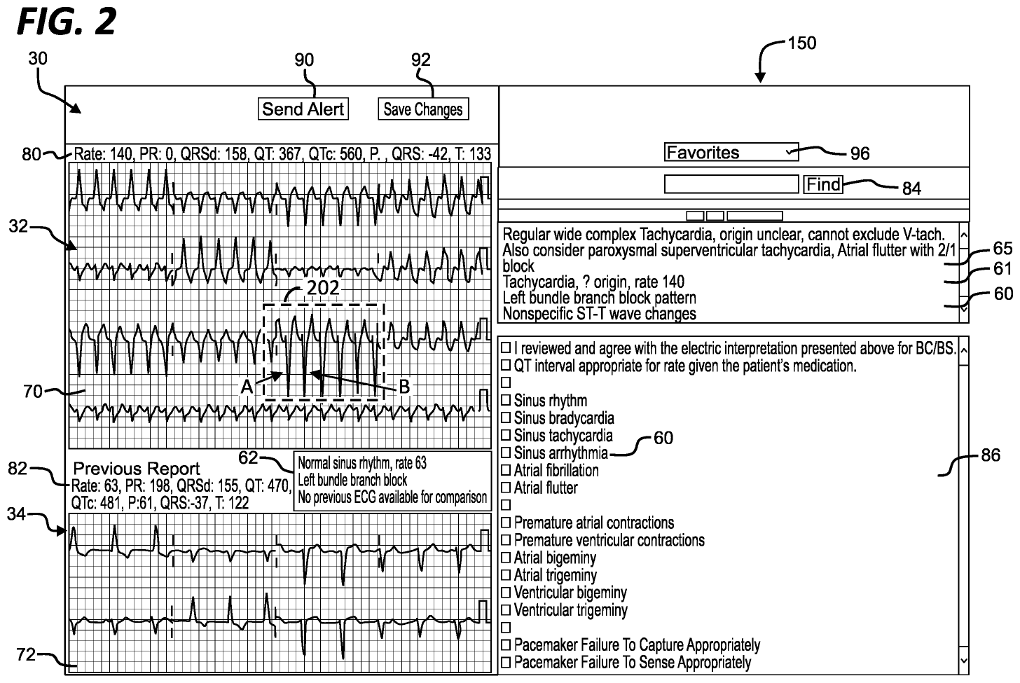

FIG. 2 illustrates an example of a graphical user interface (GUI) 150 generated by the system 10 for viewing and editing the clinical reports 30. The clinical report 30 displayed on the GUI 150 includes a current electrocardiogram waveform 70 and current physiological measurements 80 such as heart rate, pulse rate, blood oxygen saturation (SpO2), ECG QRS duration (QRSd), QT interval, and the like. The GUI 150 can be displayed on a workstation 52 used by the clinician 54. Additionally, the GUI 150 is displayed on the cardiograph device 22. In some instances, the GUI 150 is displayed on a display device 12 of the system 10.

The clinical report 30 includes interpretive statements 60 generated from an algorithm that analyzes the current electrocardiogram waveform 70 and the current physiological measurements 80. The interpretive statements 60 include diagnostic classifications of the state and behavior of the heart as determined from the current electrocardiogram waveform 70 and the current physiological measurements 80. The diagnostic classifications can be stored in an interpretive statements database 14. In some examples, the algorithm is a machine learning algorithm that streamlines an ECG overread process by introducing artificial intelligence into interpretive algorithms to complement clinical workflows.

Upon receiving the clinical report 30, the system 10 may access a patient file 17 and retrieve a previous clinical report 34 for the patient 24. The previous clinical report 34 may include a previous electrocardiogram waveform 72 and previous physician edited interpretive statements 62. As shown in FIG. 1, the system 10 can include a patient database 16 that stores the patient file 17. The system 10 can communicate with a hospital patient records management system 56 to access the patient file 17, when the patient file 17 is stored in a separate system. The system 10 can also store a new clinical report 32 that includes the current electrocardiogram waveform 70 and the current physiological measurements 80 in the patient file 17.

The GUI 150 allows the clinician to view the new clinical report 32 and the previous clinical report 34 side-by-side, and to edit the interpretive statements 60. The GUI 150 displays the previous electrocardiogram waveform 72, previous measurements 82, and previous physician edited interpretive statements 62 from the previous clinical report 34.

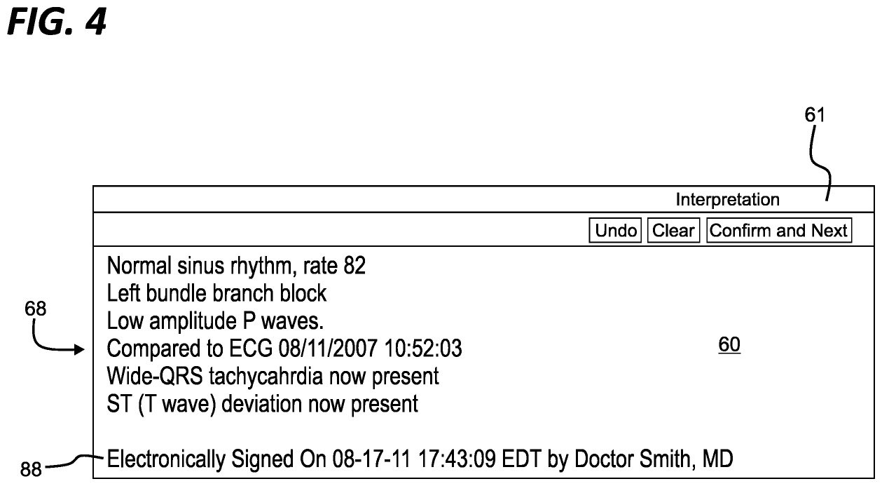

The GUI 150 further displays the new clinical report 32, which includes the current electrocardiogram waveform 70, the current physiological measurements 80, and the interpretive statements 60 which include comparison interpretive statements 65 displayed in an interpretation box 61. The clinician 54 may correct the interpretive statements 60 in the interpretation box 61 and resulting edited interpretive statements 68 (shown in FIG. 4).

The previous physician edited interpretive statements 62 include stored edits by the clinician 54 (or a different clinician) to the previous clinical report 34. The system 10 maps the previous physician edited interpretive statements 62 into one or more diagnostic codes of a structured data format to put the previous physician edited interpretive statements 62 into a format usable with a serial comparison algorithm. Each diagnostic code may uniquely identify a medical state. When no previous clinical report 34 is found, the system 10 can insert the statement “No previous report is available for comparison” into the interpretive statements 60.

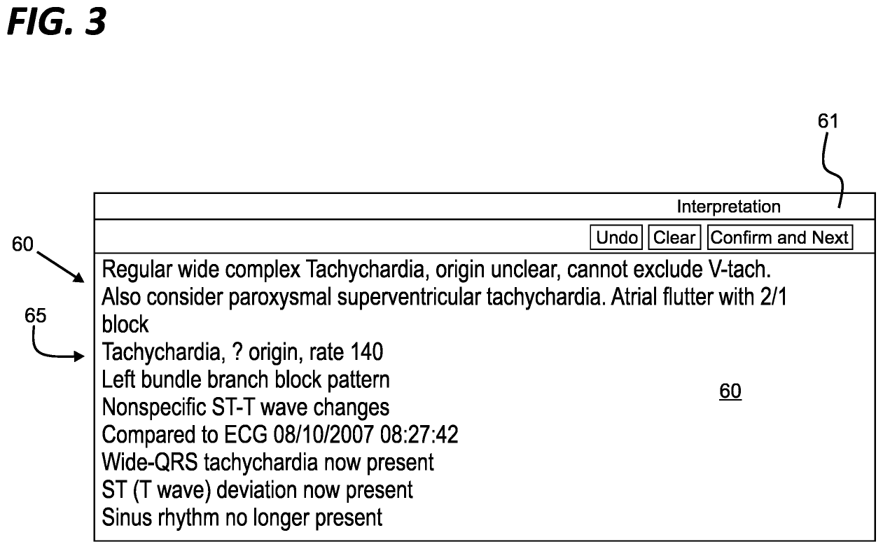

FIG. 3 illustrates in more detail an example of an interpretation box 61 that can be displayed in the GUI 150. In this example, the interpretation box 61 includes interpretive statements 60 and comparison interpretive statements 65 that are pre-populated. The interpretation box 61 is a free form text input box that permits the clinician 54 to edit one or more of the interpretive statements 60 and comparison interpretive statements 65, delete one or more of the interpretive statements 60 and comparison interpretive statements 65, and add one or more interpretive statements 60 and comparison interpretive statements 65.

FIG. 4 illustrates in more detail an example of an interpretation box 61 after the clinician 54 has edited the interpretive statements 60 to create an updated interpretation of the physiological data that includes edited interpretive statements 68. The clinician 54 may create an electronic signature 88 to sign the edited interpretive statements 68.

The system 10 may perform the serial comparison algorithm which includes an automated serial comparison of the previous clinical report 34 and the new clinical report 32 to generate the comparison interpretive statements 65. The serial comparison algorithm includes determining the diagnostic codes of the interpretive statements 60 from both the previous clinical report 34 and the new clinical report 32. The current electrocardiogram waveform 70 and current physiological measurements 80 may be examined using the diagnostic codes to determine whether waveform changes have occurred in those categories.

When the new clinical report 32 includes an interpretive statement 60 corresponding to a diagnostic code not present in the previous clinical report 34, a modifier “now present” may be added to the interpretive statement 60 to generate the comparison interpretive statement 65. When the previous clinical report 34 includes an interpretive statement 60 and there is no interpretive statement 60 in that category in the new clinical report 32, a modifier “no longer present” is added to the interpretive statement 60 to generate the comparison interpretive statement 65. Otherwise, when no significant waveform changes are detected for a diagnostic code, the system 10 adds a modifier “remains” to the interpretive statement 60 to generate the comparison interpretive statement 65. Also, when additional waveform changes are detected, a “more prominent” or “less prominent” modifier may be added. Additionally, interpretive statements 60 may be added to the comparison interpretive statements 65 to identify rhythm changes, secondary rhythm changes, heart rate changes, and the like.

A search box 84 is provided allowing the clinician 54 to search for interpretive statements 60. When the search box is active, as each character of a search string is entered, the system 10 limits the interpretive statements 60 in the library box to those interpretive statements 60 matching the search string as entered at that point in time. This may permit the clinician 54 to select the interpretive statements 60 from a results list 86 without having to input a full matching search string. A categories dialog box 96 (e.g., “Favorites”) may be provided to permit the clinician 54 to limit the results list 86 to particular categories of interpretive statements 60.

The clinician 54 may input free-form text input into the interpretation box 61, or edit the text of the comparison interpretive statements 65 already present to generate edited interpretive statements 68. In some embodiments, the clinician 54 may be permitted to input free-form text using speech recognition. In other embodiments, the clinician 54 may be permitted to input free-form text using handwriting recognition. After editing, the clinician 54 may confirm the updates to the new clinical report 32 by clicking a save changes button 92.

The physician edited interpretive statements may be parsed to extract interpretive statements 60. Parsing may require the system 10 to first convert the physician edited interpretive statements into text form. For example, the system 10 may convert audio recordings of verbal physician edited interpretive statements to text. Similarly, the system 10 may convert handwriting to text. After physician edited interpretive statements are converted to text, spelling correction, grammatical correction and normalization, removal of punctuation, and the like may then normalize the resulting text. Interpretive statements 60 may then be parsed by splitting the physician edited interpretive statements into two or more sub-strings.

The system 10 classifies substrings to correspond to at least one diagnostic code of a structured data format. Classification can be accomplished by performing a lookup in the interpretive statements database 14 that includes spelling, abbreviations, and acronym variations of the interpretive statements 60. The system 10 can lookup the diagnostic code associated with the interpretive statement 60 and store the diagnostic code in the new clinical report 32 or use it to perform serial comparison to generate the comparison interpretive statements 65.

After editing, the clinician 54 confirms the updates to the new clinical report 32 by clicking a save changes button 92. After a clinician 54 has saved changes to the new clinical report 32, the system 10 checks the edited interpretive statements 68 for statements that require a critical alert 94 to be issued. If the edited interpretive statements 68 requires a critical alert 94, system 10 may remind the physician to issue a critical alert 94.

Similarly, a send alert button 90 may be provided to permit the clinician 54 to manually issue a critical alert 94. When a critical alert 94 is issued, the system 10 may then log the critical alert 94. The critical alert 94 may be logged in the patient database 16 of the system 10 or the hospital patient records management system 56. Alternatively, failure to issue a critical alert 94 may be logged to record that the critical alert 94 was affirmatively not issued.

Referring back to FIG. 1, the system 10 can receive additional physiological data in the clinical report 30, such as data collected from stress tests 40, a Holter monitoring 41, defibrillators 42, pulmonary function testing (PFT) 43, event monitoring 44, pacemaker 45, mobile telemetry 46, vital signs 47, electroencephalogram (EEG) 48, blood pressure data 49, sleep monitoring 50, and the like. The clinical report 30 may be received from the one or more connected devices, and may include interpretive statements 60 that are computer generated.

Aspects described herein are controlled by one or more controllers 15. The one or more controllers 15 may be adapted run a variety of application programs, access and store data, including accessing and storing data in associated databases, and enable one or more interactions via the user interface. The one or more controllers 15 include at least one processing device 19, and a memory device 21 storing instructions which, when executed by the at least one processing device, cause the at least one processing device to perform the functionalities described herein.

The at least one processing device 19 can include a central processing unit (CPU). The CPU can include a single microprocessor, or a plurality of microprocessors for configuring the CPU as a multi-processor system. The memory device 21 can include a main memory, such as a dynamic random access memory (DRAM) and cache, as well as a read only memory, such as a PROM, EPROM, FLASH-EPROM, or the like. The system 10 may also include any form of volatile or non-volatile memory. The main memory stores at least portions of instructions for execution by the CPU and data for processing in accordance with the executed instructions.

The one or more controllers 15 may also include one or more input/output interfaces for communications with one or more processing systems. Although not shown, one or more such interfaces may enable communications via a network, e.g., to enable sending and receiving instructions electronically. The communication links may be wired or wireless.

The one or more controllers 15 may further include input/output ports for interconnection with one or more output devices (e.g., display device 12, additional display devices including monitors and touchscreens, printers, and other output devices) and one or more input devices (e.g., keyboard, mouse, voice, touch, bioelectric devices, magnetic reader, RFID reader, barcode reader, touchscreen, motion-sensing input device, and other input devices) serving as one or more user interfaces for the controller 15. For example, the one or more controllers 15 may include a graphics subsystem to drive the output display. The links of the peripherals to the one or more controllers 15 may include wired and/or wireless connections.

Although summarized above as a PC-type implementation, those skilled in the art will recognize that the one or more controllers also encompasses systems such as host computers, servers, workstations, network terminals, and the like. Further one or more controllers may be embodied in a device, such as a mobile electronic device, like a smartphone or tablet computer. The term controller is intended to represent a broad category of components.

Hence aspects of the systems and methods provided herein encompass hardware and software for controlling the relevant functions. Software may take the form of code or executable instructions for causing a controller or other programmable equipment to perform relevant operations, where the code or instructions are carried by or otherwise embodied in a medium readable by the controller or other machine. Instructions or code for implementing such operations may be in the form of computer instruction in any form (e.g., source code, object code, interpreted code, etc.) stored in or carried by any tangible readable medium.

As used herein, terms such as computer or machine “readable medium” refer to any medium that participates in providing instructions to a processor for execution. Such a medium may take many forms. Non-volatile storage media include, for example, optical or magnetic disks, such as any of the storage devices in any computer(s) shown in the drawings. Volatile storage media include dynamic memory, such as main memory of such a computer platform. Common forms of computer-readable media therefore include for example: a floppy disk, a flexible disk, hard disk, magnetic tape, any other magnetic medium, a CD-ROM, DVD, any other optical medium, a RAM, a PROM and EPROM, a FLASH-EPROM, any other memory chip or cartridge, or any other medium from which a computer can read programming code and/or data. Many of these forms of computer readable media may be involved in carrying one or more sequences of one or more instructions to a processor for execution.

The algorithm performed on the current electrocardiogram waveform 70 and the current physiological measurements 80 is a machine learning algorithm that streamlines clinician overread process and enhance patient outcomes by introducing artificial intelligence methodology into interpretive algorithms. The algorithm can provide a more accurate, consistent, and reliable automatic interpretation of ECGs which elevates clinician confidence in identifying critical conditions, improving the recognition of artifact and prioritizing exams for review. For example, the algorithm can automatically learn to identify patterns in traditional and non-traditional ECG features, and that is capable of learning novel high level ECG patterns. The algorithm can include an ECG interpretation vocabulary better aligned with the current clinical uses of the ECG, including identifying the reason the ECG was requested, clinical and historical context of the ECG, and clinical actionability of the ECG. In some examples, the algorithm is a deep learning algorithm. In some examples, the algorithm includes a convolutional neural network (CNN). In some further examples, the algorithm includes saliency mapping. In alternative examples, the algorithm includes activation mapping. In yet further example, the algorithm can include additional types of machine learning and artificial intelligence algorithms.

The algorithm can be trained using a large number of historical digital ECGs together with objective evidence of “true” diagnosis and/or ECG interpretation. For example, depending on the prevalence of conditions and required accuracy, the algorithm can be developed using over a million historical digital ECGs. Evidence of a true diagnoses can include a signature of a human reader on a final ECG interpretation. However, it is known that human ECG readers can be biased by the original automatic interpretation, their own clinical experience, or simply make mistakes. Thus, multiple readings of the same ECG by more than one physician can be used for stronger evidence of true diagnosis. For some classes of ECG findings (e.g., those involving acute myocardial infarctions) actual and/or historical clinical data can reinforce the evidence of true diagnosis. The larger and more variate the database of historical digital ECGs is, the more useful it is for the development (training) of the algorithm given presence of imperfect true diagnosis annotations. For the validation of accuracy claims and clinical acceptability, a smaller database with strong truth annotations can be used as well.

The historical digital ECGs are de-identified to remove protected health information for compliance with Health Insurance Portability and Accountability Act of 1996 (HIPAA) privacy rules. The database further includes one or preferably more automatic interpretations as well as human readings of the historical digital ECGs. Each interpretation category can include at least 100-1000 abnormal findings in various degrees (depending on the prevalence and clinical impact of the category), as well as at least 10 times as many normal findings. The human reading is performed by a group of physicians having clinical expertise of ECG interpretation, including but not limited to cardiologists and cardiac electrophysiologists.

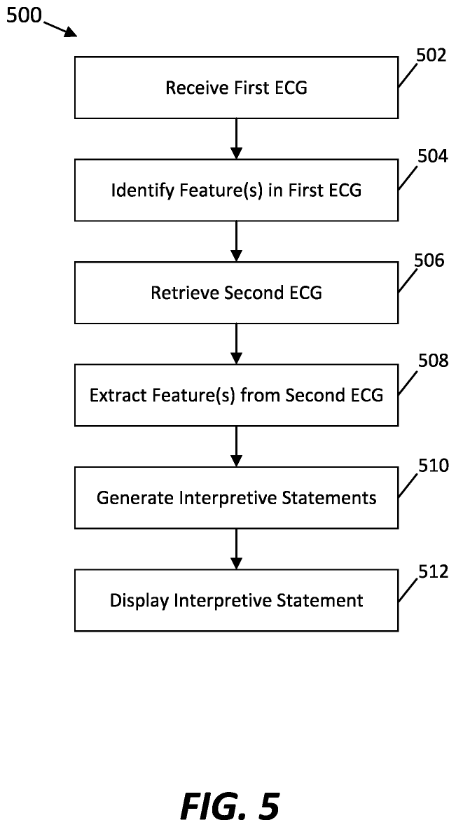

FIG. 5 illustrates an example of a method 500 of interpreting electrocardiograms. The method 500 can be performed by the system 10. The method 500 can improve the accuracy of the interpretive statements generated by the algorithm. The method 500 generates the interpretive statements based on the current electrocardiogram waveform 70 and current physiological measurements 80 and data acquired from one or more previous ECGs of the patient 24, such as the previous electrocardiogram waveform 72 from the previous clinical report 34. The interpretive statements generated in accordance with the method 500 are different from the comparison interpretive statements 65 which are determined by comparing diagnostic codes of the interpretive statements between the new clinical report 32 and the previous clinical report 34. Instead, the interpretive statements generated by the method 500 modify the primary computerized interpretation such that the method 500 refines or replaces an interpretation of the current electrocardiogram waveform 70 based on the previous electrocardiogram waveform 72.

As shown in FIG. 5, the method 500 includes an operation 502 of receiving a first electrocardiogram waveform. As an illustrative example, the first electrocardiogram waveform is the current electrocardiogram waveform 70 from the new clinical report 32. The first electrocardiogram waveform can be recorded by the cardiograph device 22. In some examples, the first electrocardiogram waveform is received by the system 10 via the network interface 11.

Next, the method 500 includes an operation 504 of identifying one or more features in the first electrocardiogram waveform. Operation 504 can include performing the algorithm on the first electrocardiogram waveform to identify one or more portions of the first electrocardiogram waveform as abnormal. In examples where the algorithm is a machine learning algorithm, the algorithm can be hosted on a cloud-based server. In some examples, operation 504 includes performing saliency mapping for identifying the one or more portions of the first electrocardiogram waveform as abnormal.

Next, the method 500 includes an operation 506 of retrieving one or more second electrocardiogram waveforms, the one or more second electrocardiogram waveforms recorded at points in time prior to the first electrocardiogram waveform. As an illustrative example, the one or more second electrocardiogram waveforms can include the previous electrocardiogram waveform 72 from the previous clinical report 34. In some examples, operation 506 includes retrieving a plurality of the second electrocardiogram waveforms. The one or more second electrocardiogram waveforms can be recorded by the cardiograph device 22, or by other cardiograph devices. In some examples, the one or more second electrocardiogram waveforms are received by the system 10 via the patient file 17 stored in the patient database 16.

Next, the method 500 includes an operation 508 of extracting the one or more features (identified in the first electrocardiogram waveform) from the one or more second electrocardiogram waveforms. In some examples, the one or more features extracted from the one or more second electrocardiogram waveforms include at least one portion of the one or more second electrocardiogram waveforms identified as abnormal. In some examples, operation 508 can include performing the algorithm on the one or more second electrocardiogram waveforms to extract the one or more features, including the at least one portion identified as abnormal.

Next, the method 500 includes an operation 510 of generating one or more interpretive statements based on the one or more features identified in the first electrocardiogram and the one or more features extracted from the one or more second electrocardiogram waveforms. The one or more interpretive statements generated in operation 510 are different from the comparison interpretive statements 65 because the interpretive statements generated in operation 510 include a primary computerized interpretation based on both the current electrocardiogram waveform 70 and the one or more previous electrocardiogram waveforms 72. The method 500 provides a comparison between at least two electrocardiogram waveforms recorded at different points in time to determine whether there are abnormalities in a first electrocardiogram waveform, and whether the abnormalities are “normal” for the patient.

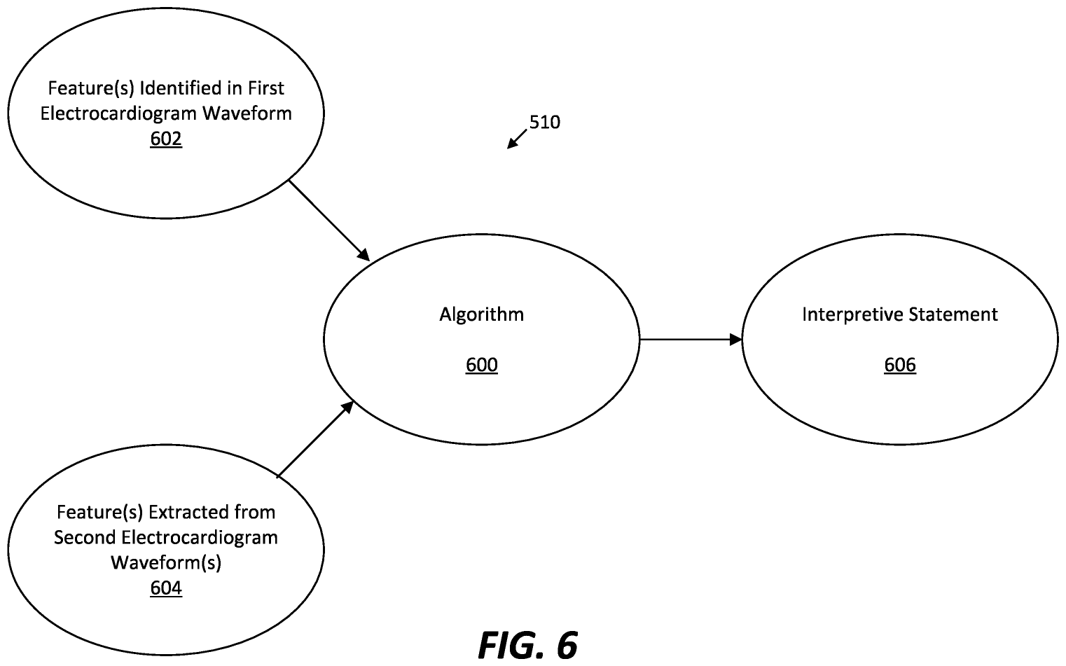

FIG. 6 schematically illustrates an example of a process flow diagram for an algorithm 600 that can be executed in the operation 510 of the method 500. In this example, the algorithm 600 receives as inputs the one or more features identified in the first electrocardiogram waveform 602 and the one or more features extracted from the one or more second electrocardiogram waveforms 604 to generate an interpretive statement 606 as an output.

The algorithm 600 does not compare diagnostic codes between the first and second electrocardiogram waveforms to describe changes between the first and second electrocardiogram waveforms such as whether an abnormality “remains” or “is no longer present”, as is done to compute the comparison interpretive statements 65. Instead, the algorithm 600 generates the interpretive statement 606 as a primary computerized interpretation of the first electrocardiogram waveform based on the one or more features identified in the first electrocardiogram waveform 602 and the one or more features extracted from the one or more second electrocardiogram waveforms 604. The algorithm 600 is more complex than comparing diagnostic codes to describe changes between the first and second electrocardiogram waveforms because, instead, the algorithm compares portions of the first electrocardiogram waveform with portions of the one or more second electrocardiogram waveforms.

In some examples, the algorithm 600 includes a machine learning algorithm such as a deep learning algorithm, a convolutional neural network (CNN), and the like. In some examples, the one or more abnormalities identified in the first electrocardiogram waveform 602 and/or the features extracted from the one or more second electrocardiogram waveforms 604 are used by the algorithm 600 to train a boosted decision tree model. In examples where the algorithm 600 includes a machine learning algorithm or artificial intelligence methodology, the algorithm 600 is hosted on a cloud-based server that communicates with the cardiograph device 22. In some examples, the system 10 is a cloud-based server that hosts the algorithm 600.

In some examples, the algorithm 600 can generate confidence levels for the interpretive statements 606 based on the one or more features extracted from the one or more second electrocardiogram waveforms. As an illustrative example, when ST levels in the first electrocardiogram waveform are different from the ST levels in a second electrocardiogram waveform, a higher confidence of acute myocardial infarction (AMI) may be provided.

The algorithm 600 can additionally evaluate changes in Q wave area, changes in “hyperacutivity” of T waves (i.e., calculating a score to quantify an amplitude of a hyperacute T wave, and calculating changes in that score), and changes in a “subtle ST elevation (STE)” score. Additional evaluations that can be performed by the algorithm 600 are contemplated.

In some examples, the algorithm 600 can include determining a first Q duration sum from a set of leads that are used to record the first electrocardiogram waveform, determining a second Q duration sum from the set of leads that are used to record a second electrocardiogram waveform, and generating an interpretive statement 606 based on a difference between the first and second Q duration sums (i.e., “delta QDsum”). As an example, the absence of Q waves in leads V5-6 is considered abnormal, and can be due to Left Bundle Branch Block (LBBB).

In some further examples, the algorithm 600 can also include determining a first ST segment sum from a set of leads used to record the first electrocardiogram waveform, determining a second ST segment sum from the set of leads that are used to record a second electrocardiogram waveform, and generating an interpretive statement 606 based on a difference between the first and second ST segment sums (i.e., “delta STsum”). The delta STsum can be used to identify an artery responsible for inferior acute myocardial infarction.

The algorithm 600 can also include measuring a difference in median beats between the first electrocardiogram waveform and a second electrocardiogram waveform, and generating an interpretive statement 606 based on a difference in median beats (i.e., “delta median beat”).

Referring back to FIG. 5, in some examples, the method 500 can further includes an operation of indicating whether an interpretive statement is modified based on the features extracted from the one or more second electrocardiogram waveforms. For example, the method 500 can include displaying whether the one or more interpretive statements are added, removed, or replaced based on the features extracted from the one or more second electrocardiogram waveforms 604. As another example, the method 500 can display a notation on the GUI 150 that an interpretive statement is added, removed, or replaced based on the features extracted from the one or more second electrocardiogram waveforms 604. The notation displayed on the GUI can provide further context, allowing the clinician to follow up and look at the one or more second electrocardiogram waveforms 604 to verify the fidelity of the interpretive statement.

In some examples, the method 500 can include generating the one or more interpretive statements without using features extracted from a second electrocardiogram waveform when the point in time at which the second electrocardiogram waveform is recorded prior to the first electrocardiogram waveform is less than a threshold. As an illustrative example, the threshold is five days. As a further illustrative example, the algorithm 600 may utilize the time interval between the first and second electrocardiogram waveforms such that when classifying for acute myocardial infarctions, the features extracted from the one or more second electrocardiogram waveforms 604 may be included only when older than 5 days.

In some examples, the method 500 can further include an operation 512 of displaying the one or more interpretive statements on the cardiograph device 22. For example, the method 500 when performed on the system 10 can include causing the system 10 to display the one or more interpretive statements on the cardiograph device 22 via the network interface 11. As discussed above, in some examples, the system 10 is a cloud-based server that hosts the algorithm 600, which includes artificial intelligence and/or machine learning algorithms. The algorithm 600 provides a technical effect and/or practical application of improved computerized interpretation of electrocardiograms that is presented back to the cardiograph device 22, which may help avoid unnecessary catheterization labs, interruptions in patient care, and unnecessary urgent reviews by cardiologists, thus improving patient care and saving healthcare resources.

Advantages of the method 500 can include, without limitation, improving the accuracy of interpreting electrocardiogram waveforms. Also, it can be advantageous to indicate in the interpretive statements transmitted back to the cardiograph device 22 which statements were modified in view of the prior electrocardiogram waveforms. For example, this can at least tell the technician that there is something relevant in the prior electrocardiogram waveforms, and can give the technician additional confidence in the fidelity of the interpretive statements when deciding whether to request urgent review by a cardiologist

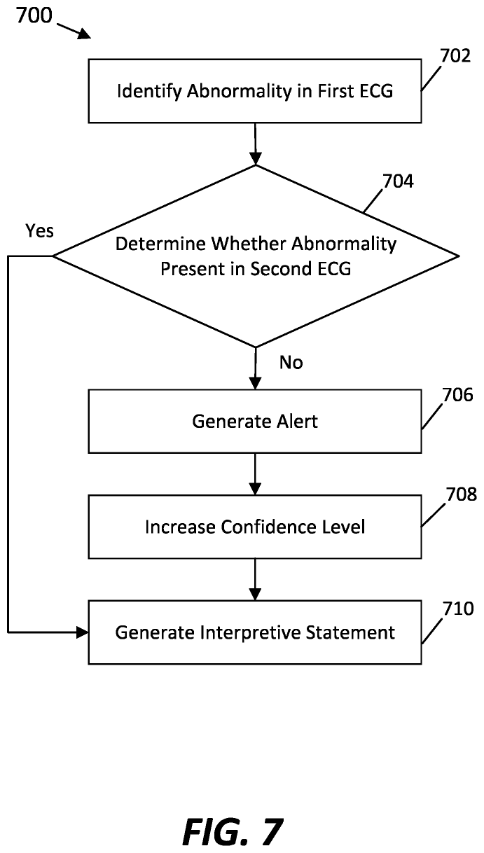

FIG. 7 schematically illustrates another example of a method 700 of interpreting electrocardiograms. The method 700 can be performed by the system 10. In some examples, the method 700 complements the method 500 such that the method 700 can be performed on the system 10 in addition to the method 500 (either simultaneously, before, or after the method 500).

As shown in FIG. 7, the method 700 includes an operation 702 of identifying an abnormality in the first electrocardiogram, followed by an operation 704 of determining whether the abnormality is present in the one or more second electrocardiogram waveforms. When the abnormality is present in the one or more second electrocardiogram waveforms (i.e., “Yes” in operation 704), the method 700 can proceed to an operation 710 of generating an interpretive statement because the abnormality may be a normal condition of the patient 24.

When the abnormality is not present in the one or more second electrocardiogram waveforms (i.e., “No” in operation 704), the method 700 can proceed to operation 706 of generating an alert. As an illustrative example, when elevated ST levels in the first electrocardiogram are not present in the one or more second electrocardiogram waveforms, this can be indicative of acute myocardial infarction (AMI) such that an alert is automatically generated in operation 706. In some examples, the method 700 can include a further operation 708 of increasing a confidence level of an interpretive statement that is indicative of AMI when the abnormality (e.g., elevated ST levels) is not present in the one or more second electrocardiogram waveforms (i.e., “No” in operation 704).

The following are examples of serial comparison algorithms that can be performed to reduce false positive detection of AMI in various regions of the heart such as inferior wall AMI, anterior AMI, lateral wall AMI, and septal AMI. Such algorithms can be performed as part of the method 500 such as to generate interpretive statements (i.e., operation 510) based on the one or more features identified in the first electrocardiogram and the one or more features extracted from the one or more second electrocardiogram waveforms. Such algorithms can also be performed as part of the method 700 such as to generate interpretive statements (i.e., operation 710) after an abnormality is identified in the first electrocardiogram.

For each region of the heart discussed below, the serial comparison algorithms were made to a newest available prior ECG that is at least 5 days older than a current ECG. The following example algorithms confirm whether MI detected in the current ECG is old by checking for supporting evidence in the prior ECG. When such evidence is found, the interpretative statement for the current ECG is changed from “new” (i.e., acute) MI to “old” MI to reduce false positive detection of AMI. As an illustrative example, the serial comparison algorithms can reduce AMI false positives by about 25%. This is clinically meaningful since AMI requires sending a patient to a catheterization laboratory, whereas old MI requires no immediate action. A catheterization laboratory, commonly referred to as a “cath lab,” is an examination room in a hospital or clinic with diagnostic imaging equipment used to visualize the arteries of the heart and the chambers of the heart and treat any abnormality found.

To reduce false positive detection of inferior wall AMI, the II, III and aVF leads are checked to determine whether any of these leads meet at least one of the following criteria: (1) STm in the current ECG >50 uV than in the prior ECG; or (2) current ECG has a bowed down ST segment while the prior ECG has a bowed-up ST segment and an ST <75 uV; or (3) current ECG has q wave amplitude ≥100 uV and equivalent q wave duration ≥35 ms and none of the prior ECGs 5 days or older have a q wave amplitude and equivalent duration larger than 100 uV and 35 ms. When none or only one of the II, III and aVF leads meets any of the above criteria, the MI is likely old, and the interpretive statement is modified accordingly.

To reduce false positive detection of anterior AMI, the V3 and V4 leads are checked to determine whether any of these leads meet at least one of the following criteria: (1) STm in the current ECG >50 uV than in the prior ECG; or (2) current ECG has a bowed down ST segment while the prior ECG has a bowed-up ST segment and an ST <150 uV; or (3) current ECG has q wave amplitude ≥50 and equivalent q wave duration ≥35 ms and none of the prior ECGs 5 days or older have a q wave amplitude and equivalent duration larger than 100 uV and 35 ms, respectively, in the V2-V4 leads (when testing the V3 lead) or the V3-V5 leads (when testing the V4 lead). When neither of the V3 and V4 leads meet any of the above criteria, the MI is likely old, and the interpretive statement is modified accordingly.

To reduce false positive detection of lateral wall AMI, the I, V5 and V6 leads are checked to determine whether any of these leads meet at least one of the following criteria: (1) STm in the current ECG >50 uV than in the prior ECG; or (2) current ECG has an inverted T wave (for the I lead) or a bowed down ST segment (for the V5 or V6 leads) while the prior ECG has an upward T wave (for the I lead) or a bowed-up ST segment (for the V5 and V6 leads) and an ST <150 uV; or (3) current ECG has q wave amplitude ≥50 uV and equivalent q wave duration ≥35 ms and none of the prior ECGs 5 days or older have a q wave amplitude and equivalent duration larger than 50 uV and 35 ms, respectively. When none or only one of the I, V5 and V6 leads meets any of the above the criteria, the MI is likely old, and the interpretive statement is modified accordingly.

To reduce false positive detection of septal AMI, the V2 lead is checked to determine whether the V2 lead meets at least one of the following criteria: (1) STm in the current ECG >50 uV than in the prior ECG; or (2) current ECG has an inverted T wave while the prior ECG has an upward T wave and an ST <50 uV; or (3) current ECG has q wave amplitude ≥50 uV and equivalent q wave duration ≥35 ms and none of the prior ECGs 5 days or older have a q wave amplitude and equivalent duration larger than 50 uV and 35 ms, respectively. When the V2 lead does not meet any of the above criteria, the MI is likely old, and the interpretive statement is modified accordingly.

The algorithm 600 can consume computing resources that are typically not available on cost-optimized cardiographs such as the cardiograph device 22 (see FIG. 1). For example, the algorithm 600 can include boosted decision trees trained on large electrocardiogram databases that yield models requiring large amounts of computer memory and/or processing capacity. In further examples, the algorithm 600 can include saliency mapping that produce images from electrocardiogram waveforms, and use convolutional neural networks (CNNs) for classification, which also require large amounts of computer memory and/or processing capacity.

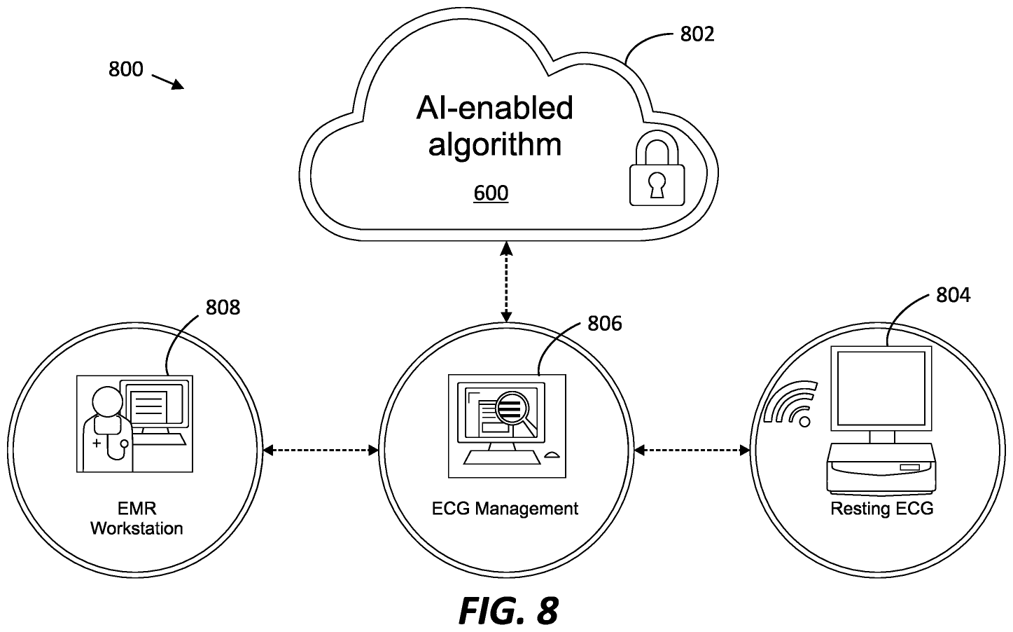

FIG. 8 schematically illustrates an example of a system 800 that can be used to implement aspects of the methods and algorithms described above. In this example, the system 800 includes a cloud-based server 802 that hosts the algorithm 600 for generating outputs that can include one or more interpretive statements 606. As described above, the algorithm 600 can include artificial intelligence methodologies and/or machine learning algorithms that can improve the accuracy of the one or more interpretive statements 606.

As shown in FIG. 8, the system 800 further includes a cardiograph device 804, an ECG workstation 806 installed with ECG management software and operatively connected to the cardiograph device 804, and an EMR workstation 808 operatively connected to the ECG workstation 806. In this example, a clinician orders an electrocardiogram for a patient using the EMR workstation 808. The order is transmitted to the ECG workstation 806 where a technician can view the order, and the technician subsequently performs an ECG examination of the patient using the cardiograph device 804. The cardiograph device 804 transmits test results including an electrocardiogram waveform back to the ECG management software installed on the ECG workstation 806. The ECG management software then communicates the test results including the electrocardiogram waveform to the cloud-based server 802 where the algorithm 600 is executed to generate the one or more interpretive statements 606 for display on the ECG workstation 806. In some examples, the one or more interpretive statements 606 can also be displayed on the cardiograph device 804 via a connection with the ECG workstation 806.

In some examples, a specialist such as a cardiologist can access a clinical report 30 via the ECG management software to view the test results including the electrocardiogram waveform, and edit the one or more interpretive statements 606 to generate one or more edited interpretive statements 68, as described above. The clinical report 30 once confirmed and/or signed by the specialist is stored to the patient file 17, and can be accessed by the clinician who ordered the electrocardiogram via the EMR workstation 908. In this example, the cardiograph device 804 is vendor agnostic such that any resting ECG device can be used in this workflow.

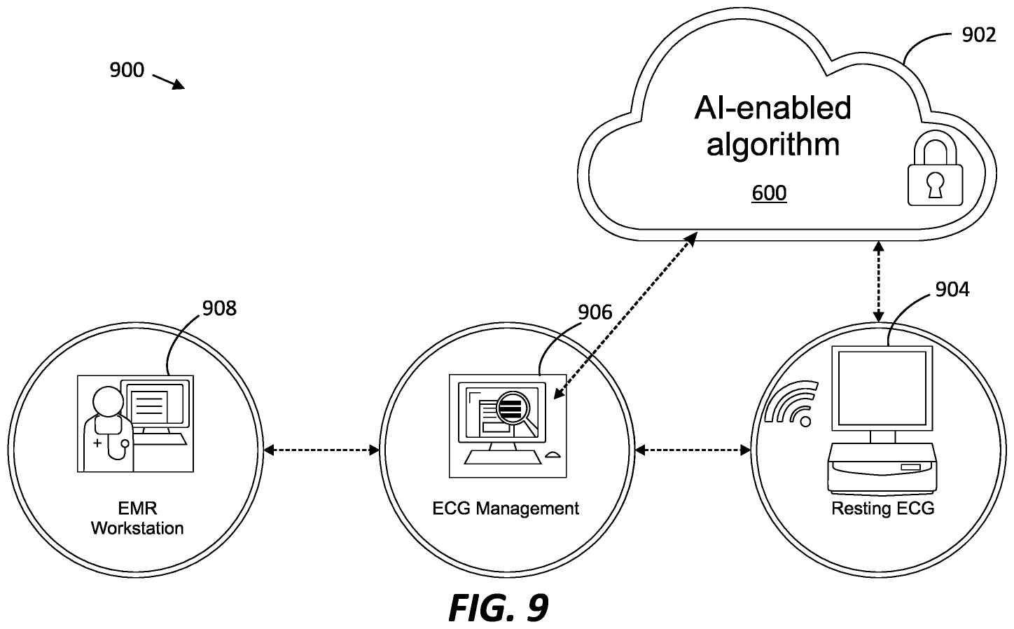

FIG. 9 schematically illustrates an example of a system 900 that can be used to implement aspects of the methods and algorithms described above Like in the prior example described above, the system 900 includes a cloud-based server 902 hosting the algorithm 600 for generating outputs that can include one or more interpretive statements 606.

The system 900 further includes a cardiograph device 904, an ECG workstation 906 installed with ECG management software and operatively connected to the cardiograph device 904, and an EMR workstation 908 operatively connected to the ECG workstation 906. In this example, a clinician orders an electrocardiogram for a patient using the EMR workstation 908. The order is transmitted to the ECG workstation 906 where a technician can view the order, and then perform an ECG examination of the patient using the cardiograph device 904.

The cardiograph device 904 transmits test results including an electrocardiogram waveform to the cloud-based server 902 where the algorithm 600 is executed to generate the one or more interpretive statements 606 for display on the cardiograph device 904. In some examples, the cardiograph device 904 transmits a clinical report 30 including the electrocardiogram waveform and interpretive statements 606 to the ECG workstation 906. Alternatively, the cloud-based server 902 can transmit the clinical report 30 including the electrocardiogram waveform and interpretive statements 606 to the ECG workstation 906.

In some examples, a specialist such as a cardiologist can access the clinical report 30 via the ECG management software to view the test results including the current electrocardiogram waveform, and edit the one or more interpretive statements 606 to generate one or more edited interpretive statements 68, as described above. Thereafter, the clinical report 30 once confirmed and/or signed by the specialist is stored to the patient file 17 can be accessed via the EMR workstation 908. In this example, the ECG management software installed on the ECG workstation 906 is vendor agnostic such that any ECG management software can be used.

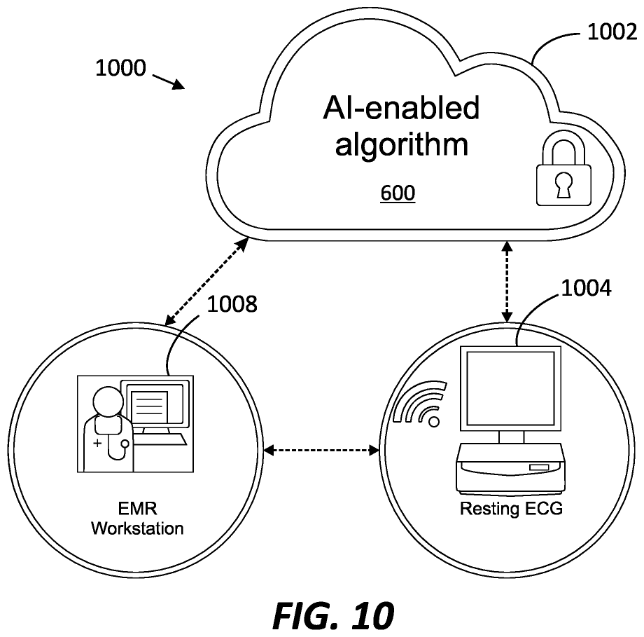

FIG. 10 schematically illustrates an example of a system 1000 that can be used to implement aspects of the methods and algorithms described above. Like in the prior examples described above, the system 1000 includes a cloud-based server 1002 hosting the algorithm 600 for generating outputs that can include one or more interpretive statements 606.

The system 1000 further includes a cardiograph device 1004 operatively connected to an EMR workstation 1008 and the cloud-based server 1002. In this example, a clinician orders an electrocardiogram for a patient using the EMR workstation 1008. The order is transmitted to the cardiograph device 1004 where a technician can view the order, and then perform an ECG examination of the patient using the cardiograph device 1004. In this example, the cardiograph device 1004 transmits test results including a current electrocardiogram waveform to the cloud-based server 1002 where the algorithm 600 is executed to generate the one or more interpretive statements 606 for display on the cardiograph device 1004. The cardiograph device 1004 can then transmit a clinical report 30 including the current electrocardiogram waveform and the one or more interpretive statements 606 to the EMR workstation 1008. Alternatively, the cloud-based server 1002 can transmit the clinical report 30 including the current electrocardiogram waveform and the one or more interpretive statements 606 to the EMR workstation 1008. In this example, the system 1000 does not include a dedicated ECG workstation.

Advantages of the systems 800, 900, 1000 can include providing computational infrastructure allowing the algorithm 600 to utilize advanced artificial intelligence and machine learning techniques to provide accurate computerized interpretation at the point of electrocardiogram acquisition (i.e., the cardiograph devices 804, 904, 1004), and on dedicated ECG workstations. Further advantages of the systems 800, 900, 1000 can include providing access to a database of prior electrocardiograms for the algorithm 600 to improve the accuracy of the one or more interpretive statements 606 generated from the electrocardiograms measured by the cardiograph devices 804, 904, 1004. Additional advantages of the systems 800, 900, 1000 can include avoiding costly upgrades to the cardiograph devices 804, 904, 1004 to enable them to run more advanced algorithms. Also, the computing resources of the cloud-based servers 802, 902, 1002 are much more powerful than the processing devices on the cardiograph devices 804, 904, 1004, and can produce an ECG interpretation faster than traditional cardiograph devices (even when considering transmission delays to and from the cloud-hosted algorithm).

The various embodiments described above are provided by way of illustration only and should not be construed to be limiting in any way. Various modifications can be made to the embodiments described above without departing from the true spirit and scope of the disclosure.

What is claimed is:

1. A system for interpreting electrocardiograms, the system comprising:
at least one processing device; and
a memory device storing instructions which, when executed by the at least one processing device, cause the at least one processing device to:
receive a first electrocardiogram waveform;
identify a feature in the first electrocardiogram waveform;
retrieve one or more second electrocardiogram waveforms, the one or more second electrocardiogram waveforms being recorded at a point in time prior to the first electrocardiogram waveform;
extract the feature from the one or more second electrocardiogram waveforms; and
generate one or more interpretive statements based on a comparison of the feature identified in the first electrocardiogram waveform and the feature extracted from the one or more second electrocardiogram waveforms.

2. The system of claim 1, wherein the feature identified in the first electrocardiogram waveform includes an abnormality.

3. The system of claim 1, wherein the instructions, when executed by the at least one processing device, further cause the at least one processing device to:
generate confidence levels for the one or more interpretive statements based on the feature extracted from the one or more second electrocardiogram waveforms.

4. The system of claim 1, wherein the instructions, when executed by the at least one processing device, further cause the at least one processing device to:
indicate whether the one or more interpretive statements are modified based on the feature extracted from the one or more second electrocardiogram waveforms.

5. The system of claim 1, wherein the instructions, when executed by the at least one processing device, further cause the at least one processing device to:
generate the one or more interpretive statements without using the feature extracted from the one or more second electrocardiogram waveforms when the point in time prior to the first electrocardiogram waveform is less than a threshold.

6. The system of claim 5, wherein the threshold is five days.

7. The system of claim 1, wherein the instructions, when executed by the at least one processing device, further cause the at least one processing device to:
display the one or more interpretive statements on a cardiograph device.

8. The system of claim 1, wherein the instructions, when executed by the at least one processing device, further cause the at least one processing device to:
perform saliency mapping for identifying the feature in the first electrocardiogram waveform.

9. The system of claim 1, wherein the instructions, when executed by the at least one processing device, further cause the at least one processing device to:
identify an abnormality in the first electrocardiogram waveform;
determine whether the abnormality is present in the one or more second electrocardiogram waveforms; and
generate an alert when the abnormality is not present in the one or more second electrocardiogram waveforms.

10. The system of claim 9, wherein the instructions, when executed by the at least one processing device, further cause the at least one processing device to:
increase a confidence level of at least one of the one or more interpretive statements when the abnormality is not present in the one or more second electrocardiogram waveforms.

11. The system of claim 1, wherein the instructions, when executed by the at least one processing device, further cause the at least one processing device to:
determine a first Q duration sum from a set of leads that are used to record the first electrocardiogram waveform;
determine a second Q duration sum from the set of leads that are used to record the one or more second electrocardiogram waveforms; and
generate an interpretive statement based on a difference between the first and second Q duration sums.

12. The system of claim 1, wherein the instructions, when executed by the at least one processing device, further cause the at least one processing device to:
determine a difference in a sum of ST segments between the first electrocardiogram waveform and the one or more second electrocardiogram waveforms; and
generate an interpretive statement based on the difference in the sum of ST segments.

13. The system of claim 1, wherein the instructions, when executed by the at least one processing device, further cause the at least one processing device to:
measure a difference in median beats between the first electrocardiogram waveform and the one or more second electrocardiogram waveforms; and
generate an interpretive statement based on the difference in median beats.

14. The system of claim 1, wherein the feature in the first electrocardiogram waveform is identified by a machine learning algorithm hosted on a cloud-based server.

15. A method of interpreting electrocardiograms, the method comprising:
receiving a first electrocardiogram waveform;
identifying a feature in the first electrocardiogram waveform;
retrieving one or more second electrocardiogram waveforms, the one or more second electrocardiogram waveforms being recorded at a point in time prior to the first electrocardiogram waveform;
extracting the feature from the one or more second electrocardiogram waveforms; and
generating one or more interpretive statements based on a comparison of the feature identified in the first electrocardiogram waveform and the feature extracted from the one or more second electrocardiogram waveforms.

16. The method of claim 15, further comprising:
generating confidence levels for the one or more interpretive statements based on the feature extracted from the one or more second electrocardiogram waveforms.

17. The method of claim 15, further comprising:
indicating whether the one or more interpretive statements are modified based on the feature extracted from the one or more second electrocardiogram waveforms.

18. The method of claim 15, further comprising:
identifying an abnormality in the first electrocardiogram waveform;
determining whether the abnormality is present in the one or more second electrocardiogram waveforms; and
generating an alert when the abnormality is not present in the one or more second electrocardiogram waveforms.

19. The method of claim 18, further comprising:
increasing a confidence level of at least one of the one or more interpretive statements when the abnormality is not present in the one or more second electrocardiogram waveforms.

20. A computer-readable data storage medium comprising software instructions that, when executed, cause at least one computing device to:
receive a first electrocardiogram waveform;
identify a feature in the first electrocardiogram waveform;
retrieve one or more second electrocardiogram waveforms, the one or more second electrocardiogram waveforms being recorded at a point in time prior to the first electrocardiogram waveform;
extract the feature from the one or more second electrocardiogram waveforms; and
generate one or more interpretive statements based on a comparison of the feature identified in the first electrocardiogram waveform and the feature extracted from the one or more second electrocardiogram waveforms.
